(19) United States
(12) Patent Application Publication
(10) Pub. No.: US 2013/0244280 A1
(43) Pub. Date: Sep. 19, 2013

(54) EXPRESSION VECTOR FOR HIGH LEVEL EXPRESSION OF RECOMBINANT PROTEINS

(71) Applicants: Aashini Parikh, Gujarat (IN); Arun Singh, Gujarat (IN); Sanjeev Kumar Mendiratta, Gujarat (IN); Ajit K Gupta, Gujarat (IN); Mansi Jakhade, Gujarat (IN)

(72) Inventors: Aashini Parikh, Gujarat (IN); Arun Singh, Gujarat (IN); Sanjeev Kumar Mendiratta, Gujarat (IN); Ajit K Gupta, Gujarat (IN); Mansi Jakhade, Gujarat (IN)

(73) Assignee: CADILA HEALTHCARE LIMITED, Ahmedabad (IN)

(21) Appl. No.: 13/877,931

(22) PCT Filed: Oct. 10, 2011
(86) PCT No.: PCT/IN11/00703

§ 371 Date: May 20, 2013

(30) Foreign Application Priority Data
Oct. 8, 2010 (IN) 2806/MUM/2010

(51) Int. Cl. C12N 15/85; C12N 15/68; C12P 21/00

(52) U.S. Cl. CPC C12N 15/85; C12P 21/00; C12N 15/68
USPC 435/69.4; 435/320.1; 435/358; 435/352; 435/69.1; 435/69.6; 435/69.5

(57) ABSTRACT

The present invention provides an expression vector for the production of proteins and peptides comprising a promoter operably linked to gene of interest, TPL and VA genes I and II, matrix attachment regions (MARs)/SARs, and antibiotic marker. The vector is transfected in suitable host cell.

### FIELD OF THE INVENTION

The present invention relates to a novel expression vector for high level expression of recombinant therapeutic proteins. In particular, the present invention discloses an expression vector having a gene sequence encoding a recombinant protein and at least one operably linked expression enhancing element such as, matrix attachment region. The said vector may further comprise of other regulatory elements. In another embodiment the invention comprises mammalian cells transfected with the said expression vector.

### BACKGROUND OF THE. INVENTION

The increased demand of therapeutic proteins is primarily due to their generally highly specific target of action which results in significantly reduced and well-defined risk of toxicity compared to small molecule based drugs. Despite having all these patient-friendly qualities, most therapeutic proteins remain inaccessible to most people in the world because they continue to remain prohibitively expensive. Therefore, life saving or other important drugs, like Erythropoietin, Darbepoietin, TNKase, Etanercept, Gonadotropins, that significantly improve quality of life, and many anti-cancer monoclonal drugs like Rituximab, Trastuzumab and other therapeutic monoclonal antibodies etc., are afforded only by a very small percentage of people while a vast majority of sick people around the world cannot afford them. There is therefore an urgent need to bring down the cost of these drugs.

A large component of this high cost is associated with manufacturing them which occurs because of their low production yields. The production yield of a clone depends upon selection of several factors such as the external factors like culture conditions (media components, temperature, pH etc.) and downstream purification process, and internal factors like selection of vector and its regulatory elements like promoter, transcription or translation enhancing elements, etc and their appropriate orientation and also choice of a suitable host cell. Mammalian cell is the most promising expression system to obtain high expression of recombinant therapeutic proteins as it has a natural capacity of glycosylation. Also, post-translational modifications in such expression systems are more likely to resemble those found in human cells expressing proteins, thus rendering physiological activity. However the expression levels in eukaryotic cells are also highly dependent on another internal factor, i.e. the integration site of the recombinant expression construct comprising the gene of interest in the genome of host cell.

Recombinant expression plasmids comprising a gene of interest encoding a desired protein are routinely used to generate stable CHO transfectants or other mammalian transfectants, expressing the desired recombinant protein. The ideal system for eukaryotic overexpression would have integration of the expression cassette in the genome of a target cell at a location that permits strong and stable or long term expression. However, most current methods achieve only random integration of plasmid DNA into the genome of the host cell. It is evident from the literature that the expression levels are highly variable in clonal populations arising out of such a transfection process (Brian K Lucas et al., Nucleic Acids Research, 1996, Vol. 24, No 9, R. T. Schimke et al., Br. J. Cancer (1985), 51, 459-465, Würtele H, et al., Gene Ther. 2003 October; 10(21):1791-9. Biotechnol Prog. 2000 September-October; 16(5):710-5, Yoshikawa T et al., Biotechnol Prog. 2000 September-October; 16(5):710-5., Kim N S, et al., Biotechnol Prog. 2001 January-February; 17(1):69-75). Also, the frequency of transfectomas carrying the stably integrated recombinant gene that are capable of expressing a desired recombinant protein at high levels is quite low.

Usually a large number of stably transfected cells must be screened to identify clones which express the recombinant proteins at high levels. This is mainly believed to be due to the effects of the genomic environment of the integration site, as the mammalian genome is really large and only about 0.1% of it contains transcriptionally active sequences (Little (1993) Nature 366:204). This phenomenon of the site of integration influencing expression is called “position effect”. The position effect regulates the expression levels of intergrated gene in a positive or negative manner due to any or all of the following mechanisms—1) presence of regulatory elements near the site of integration, which may participate in regulating the expression of the integrated gene 2) the chromatin structure at the site of integration. 3) the DNA methylation activity at the site of integration. The negative impact of position effect can be as harsh as gene silencing via DNA methylation or histone deacetylation.

It is therefore highly unlikely that the technology of random plasmid integration into the genome of CHO cells will always result in the insertion of a recombinant gene into a transcriptionally active zone capable of high levels of gene expression. This means that the number of clones that need to be screened in order to find the high expressing clone would be very large.

To overcome the problem associated with random integration the discovery of systems to integrate the desired gene in site specific manner in the genome have been described in the literature and have been used with limited success. For example some groups have used certain enzymes for the site-specific recombination mediated introduction of genes of interest in transcriptionally active sites of the genome. Recombinases such as Cre and FLP perform both integration and excision with the same target sites (Sauer, B. (1994) Curr Opin. Biotechnol., 5, 521-527, Sternberg et al, J Mol Biol, 1981, 150, 467-486, Broach et al, Cell, 1980, 21, 501-508). However, although these recombinases efficiently perform integration in mammalian cell, the net integration frequency that they mediate is low because of the excessive back reaction. Therefore, the problem of stable and high expression still remains to be solved.

Traditionally, to achieve stable high producing cell lines, methods to increase the gene copy number such as methotrexate (MTX) mediated gene amplification process have been routinely done. Transfection is followed by extensive search of single cell clones having the desired phenotype. Also, levels of methotrexate are generally increased in small increments while giving sufficient time for cells to stabilize at each increment level. Thus, this process of increasing expression from transfected mammalian cells is time consuming and labor intensive.

Transcription of eukaryotic genes is regulated by a variety of cis- and trans-acting regulatory elements (Dillon et. al., (1993) Trends Genet. 9:134). Two of the best characterized cis-elements are promoters and enhancers. Promoters are DNA sequences immediately 5′ to the coding sequence of the gene. They comprise multiple binding sites for trans-acting transcription factors, forming the basic transcription apparatus. Similarly enhancers are also composed of multiple binding sites for trans-acting transcription factors but can be found far upstream or downstream of coding sequences or even within introns. These elements can also act in an orientation independent manner. Activities of promoters and enhancers can be detected in transient expression systems and they contain elements which may or may not be tissue specific.

The inventors of the present invention have already disclosed in their application WO2007017903 the combined effect of regulatory elements such as a) a CMV promoter, b) an intron, c) TPL, d) VA genes and e) a bovine growth hormone polyadenylation sequence to achieve high expression levels of recombinant proteins.

Another category of cis-acting regulatory elements are ones which are believed to regulate the chromatin structure and includes locus control regions (LCRs) [Grosveld et. al., (1987) Cell 51:975], matrix attachment regions (MARs) [Phi-Van et. al., (1990) Mol. Cell. Biol. 10:2302], scaffold attachment regions (SARs) [Gasser and Laemmli (1987) Trens Genet. 3:16], insulator elements [Kellum and Schedl (1991) Cell 64:941] and Nuclear matrix-Associating DNAs [Bode J et. al., (1992) Science 255:195].

MARs and SARs are similar enhancers in that they are able to act over long distances, but are unique in that their effects are only detectable in stably transformed cell lines or transgenic animals. LCRs are also dissimilar to other types of enhancers in that they are position and orientation dependent, and are active in a tissue specific manner.

SARs/MARs elements have been used to remove the drawback of position effects and to provide highly active genes in the expression construct. They prevent the neighbouring host cell chromatin elements from affecting the transgene expression. MARs have been isolated from regions surrounding actively transcribed genes but also from centromere and telomeric regions. They increase the expression of desired gene by regulating the transcription activity.

Several different MARs/SARs such as Drosophila Scs boundary element, hspSAP MAR, Mouse T cell receptor TCRα, Rat locus control region, β-globin MAR, Apolipoprotein B SAR element etc. have been reported from different species and different highly expressed genes in the existing literature. Most of these elements showed, low to moderate improvement in the expression levels of the desired gene in CHO cells (P. A. Girod and Nicolas Mermod, Gene Transfer and Expression in Mammalian Cell, 2003). In contrast, Chicken Lysozyme MAR (cLysMAR) was shown to have 5 to 6 fold higher expression levels as compared to controls where the MAR elements were absent (P. A. Girod and Nicolas Mermod, Gene Transfer and Expression in Mammalian Cell, 2003). Also, when the same MARs were used at both the sides flanking the expression cassette, a 4 to 5 fold further increase in expression levels is observed (P. A. Girod and Nicolas Mermod, Gene Transfer and Expression in Mammalian Cell, 2003). Thus, it is evident that cLysMAR was most promising element reported in prior art. cLysMAR is localized far upstream of the chicken lysozyme gene (Phi-Van and Stratling, EMBO, 7, 3, pp 655-64, 1988). In transformed animal cell lines, this MAR has been shown to increase the overall level of transgene expression and to decrease its position dependent variability when placed around a reporter gene (Stief et al., Nature, 341, pp 343-345, 1989). This effect has been found to extend to heterologous promoters and cells (Phi-Van et al., Molecular and Cellular Biology, 10, 5 pp 2302-2307, 1990) as well as to the tissue specificity of transgene expression (McKnight et al., 1992).

U.S. Pat. No. 7,422,874 describes the use of β-globin MAR in combination with the regulatory elements—pSV-gal or pCMV-gal promoter, MCS site and a transcriptional termination site in the PMS vector construct to increase the expression of galactosidase reporter gene, scu-PA gene and the TGF-β SRII genes. They were able to get moderate expression levels of 20 ug/million cells for β galactosidase in 88% of the clones. They were also able to generate clones for scu-PA having 4 fold more expression levels as compared to control vector construct consisting of the same regulatory elements as the above vector except MARs. When MARs and DHFR system for gene amplification were used together in expression of TGF-β SRII, they were able to generate primary clones producing 100 ng/million cells/day after transfections and 10 ug/million cells/day after several rounds of MTX mediated gene amplification upto 1 uM MTX. However the expression levels obtained in this patent are not commercially viable today for biotherapeutics proteins such as TNKase, Darbepoietin, and monoclonal antibodies.

U.S. Pat. No. 7,371,542 describes the use of β IFN S/MAR in combination with the regulatory elements—CMV Promoter, Intron, On P and Poly A in the expression vector construct to increase the expression of a LTBR-Fc (Lymphotoxin beta receptor—IgG Fc Fusion protein) and achieved a 4.5 fold improvement in expression levels in CHO Cells as compared with control vector consisting of the same regulatory elements as the above vector except MARs. They also found that use of the β IFN S/MAR in expression vector increases the expression level 6.3 fold in 293 EBNA cells using the vector pCEP-LTBR-Fc. However the expression levels were still very low (20 mg/L in 5 days). The vector

pCB_SM1_LTBR-Fc was able to give clones in 293 EBNA cells having a productivity of 40 mg/L in 9 days. But the productivity levels obtained in this patent are far less than desirable for such biotherapeutic molecules.

U.S. Pat. No. 5,731,178 describes the enhanced expression of desired gene by using the cLysMARs in vector construct comprising promoter and enhancer. They showed that the use of the cLysMAR element in stable transfections was able to improve the reporter gene CAT activities by more than 10 fold when mar element was used in combination with enhancer and promoter element over the control construct consisting of just the enhancer and the promoter, however the MAR element was not able to show any major impact by itself.

Poljak et al., (1994) Nucleic Acid Res., 22(21):4386-94) reports the increase in expression of the CAT reporter gene by about 15 fold when cLysMAR was used in combination with an SV40 Promoter and an enhancer. The cLysMAR by itself was found to be very poor in increasing the expression of the desired gene, rather it showed a slight decrease.

Thus, the above examples demonstrate the fact that the control vector constructs comprising of the standard regulatory elements known to anyone skilled in the art were not in themselves sufficient to support high expression. And further even after combination with MARs/SARs, the expression levels did not increase to those required commercially for viable production of recombinant therapeutics. Thus, a unique combination of elements was still required to achieve desired expression levels.

Therefore it becomes clear that there exists an important need in the industry to further increase the expression of therapeutic proteins to make them more and more affordable. Therefore one feels the need for a combination of elements in the expression vector that can work along with MARs in a synergistica fashion to give increased expression with different kind of target genes. The inventors of the present invention have henceforth proved that a concerted action of a unique combination of regulatory elements is required for optimal expression of a recombinant protein in a time saving manner.

U.S. Pat. No. 7,129,062 describes the co-transfection of more than 2 unlinked vectors where one vector comprises gene of interest and second one comprises cLysMARs to increase the expression of two recombinant proteins—luciferase and anti Rhesus D IgG by about 20 folds and they were also able to produce human anti Rhesus D IgG at 200 mg/L using this cotransfection strategy. However it is well reported (DNA Cloning: Mammalian systems; By David M. Glover, B. D. Hames) in the literature and also in our experience that cotransfection increases heterogeneity and variability in the transfected population. Moreover, the present invention achieved the desired yields by using single transfection.

US20080102523 describes the use of β-globin MAR for increasing the expression of beta-galactosidase by 3 fold and immunoglobulin by 6 fold as, compared to the control vector construct consisting of SV40 promoter-enhancer and/or CMV promoter, on site, and a poly A region. The above patent application achieves only a moderate increase in expression via both the MTX mediated gene amplification pressure as well as with the help of the β-globin MAR regulatory element thus making the whole process tedious and time consuming. This is in contrast with the current invention where the inventors have achieved high expression with their unique combination of regulator elements and without using any long and tedious methods like the MTX-DHFR selection method.

U.S. Pat. No. 5,888,774 describes the high expression of erythropoietin by using human apolipoprotein B SAR element and reports an expression of 1500 to 1700 IU of EPO/million cells/24 Hrs. WO2007017903 owned by the inventors describes a process to produce recombinant human erythropoietin at an expression level of 11,830 IU/ml (91 μg/ml) in a 168 hrs culture which is equivalent to 2366 to 3549 IU/10⁶ cells/24 Hrs or 18.2 to 27.3 μg/10⁶ cells/24 Hrs, which is remarkably higher than the reported values in U.S. Pat. No. 5,888,774. And the present invention further increases expression levels significantly over the vector of WO2007017903, for several therapeutic proteins.

Thus, it is still desirable to develop novel expression vectors for further increasing the productivity of eukaryotic host cells. Surprisingly, in spite of the tremendous amount of knowledge generated in this field over the last decades even today a person skilled in the art cannot simply pick and choose a combination of internal factors or regulatory elements to design an expression vector that would give considerably high expression. Further, the combination of the suitable elements to create a high expressing vector cannot be routinely extrapolated by a skilled person since the high expression of the desired gene of interest using the vector cannot be just attributed to only one element but a combination of appropriate elements are desirable to get a stable high expressing cell lines. Hence, this invention provides a solution to this problem by providing novel expression vectors that comprise of expression enhancing elements like chicken lysozyme MAR element(s) in combination with other regulatory elements such as a CMV promoter, an intron, TPL, and VA genes which have multiple roles e.g., in increasing the mRNA levels by increased transcription, of extending the life of the mRNA molecule by increasing its stability, and by increasing the translation efficiency, thus working in a synergistic manner leading to a high and stable expression of the recombinant protein in transfected mammalian host cells. The combination of these elements in the expression vector of the present invention, consisting of the expression cassette flanked by cLysMAR in a cis or trans orientation results in a stable, high expression of therapeutic proteins in transfected cell lines.

### OBJECTS OF THE INVENTION

The present invention provides an expression vector which increases the expression efficiency of the protein of interest in mammalian cells.

In one embodiment, the present invention provides a novel expression vector comprising a promoter operably linked to the gene of interest, expression enhancement elements, other regulatory elements i.e., TPL, VA I and II genes or variants thereof, a translation terminator and an antibiotic marker wherein the expression enhancement element is a chromatin attachment region.

In one embodiment, the present invention provides the novel expression vector construct for the expression of therapeutic proteins and peptides where the expression vector construct comprises the promoter operably linked to cloning sites, gene of interest, translation terminator, TPL, VA I and II genes, suitable antibiotic marker in combination with expression enhancing elements selected from MARs and/or SARs. In an alternate embodiment, the present invention provides a novel expression vector construct for the expression of therapeutic proteins and peptides where the expression vector construct comprises the promoter operably linked to cloning sites, gene of interest, translation terminator such as BGH, intron, suitable marker and optionally internal ribosomal binding site in combination with expression enhancing elements selected from MARs and/or SARs.

In one embodiment, the present invention provides the novel expression vector construct for the expression of therapeutic proteins and peptides where the expression vector construct comprises the promoter operably linked to cloning sites, gene of interest, translation terminator such as BGH, TPL, VA I and II genes, Intron, suitable antibiotic marker and optionally internal ribosomal binding site in combination with expression enhancing elements selected from MARs and/or SARs.

Yet in another embodiment the present invention provides the process for expressing gene of interest in mammalian host cell which is transfected with the expression vector according to the embodiments of the invention.

### BRIEF DESCRIPTION OF DRAWINGS

FIG. 1 depicts the vector diagram of pZRCII

FIG. 2 depicts the vector diagram of pZRC III

FIG. 3 depicts the vector diagram of pZRC III-TNK-Hyg

FIG. 4 depicts the vector diagram of pZRC III-Darbe-Hyg

FIG. 5 depicts the vector diagram of pZRC III-Etanercept-Hyg

FIG. 6 depicts the vector diagram of pZRC III-FSH α-IRES-FSH β-Hyg vector

FIG. 7 depicts the vector diagram of pZRC III-FSH β-IRES-FSH α-Hyg vector

FIG. 8 depicts the vector diagram of pZRC III-TNK-Puro

FIG. 9 depicts the vector diagram of pZRC III-DARBE-Puro

FIG. 10 depicts the vector diagram of pZRC III-FSH α-IRES-FSH β-Puro vector

FIG. 11 depicts the vector diagram of pZRC III-FSH α-IRES-FSH β-Neo vector

FIG. 12 depicts the vector diagram of pZRC III-FSH β-IRES-FSH α-Neo vector

FIG. 13 depicts the vector diagram of pZRC III-Etanercept-Neo

FIG. 14 depicts the vector diagram of pZRC III-TNK-Neo

### DESCRIPTION OF THE INVENTION

### Definition

Chromatin attachment regions are structural components of chromatin that form topologically constrained loops of DNA through their interaction with the proteinaceous nuclear matrix.

### Abbreviations Used:

| | |
|---|---|
| CMV | Cytomegalovirus |
| TPL | Tripartite Leader |
| VA genes or VA I | Virus associated RNA genes I and II |
| and II genes | |
| BGH | Bovine growth hormone |

The present invention provides a novel expression vector which increases the efficiency of expression of therapeutic proteins and peptides significantly in mammalian host cell. The novel vector further removes the drawback associated with the position effect and adding the advantage of increased transcription and translation achieved with the unique combination of regulatory elements.

In one embodiment, the present invention provides a novel expression vector comprising a promoter operably linked to the gene of interest, expression enhancement elements, TPL, VAI and II genes or variants thereof, translation terminator and an antibiotic marker wherein the expression enhancement element is a chromatin attachment region. Chromatin attachment regions are selected from MARs and SARs. In an embodiment the present invention provides an expression vector for the production of proteins and peptides which comprises promoter operably linked to gene of interest, TPL and VA genes I and II, matrix attachment regions (MARs)/SARs, translation terminator antibiotic marker.

In an alternate embodiment, the present invention provides a novel expression vector construct for the expression of therapeutic proteins and peptides where the expression vector construct comprises the promoter operably linked to cloning sites, gene of interest, translation terminator such as BGH, intron, suitable antibiotic marker and optionally internal ribosomal binding site in combination with expression enhancing elements selected from MARs and/or SARs.

In one embodiment, the present invention provides the novel expression vector construct for the expression of therapeutic proteins and peptides where the expression vector construct comprises the promoter operably linked to cloning sites, gene of interest, translation terminator such as BGH, TPL, VA I and II genes, Intron, suitable antibiotic marker and optionally internal ribosomal binding in combination with expression enhancing elements selected from MARs and/or SARs.

According to an embodiment of the present invention, the promoter is selected from the group consisting of CMV promoter, SV40 promoter, adenovirus promoter, Beta actin promoter, metallothionin promoters or other prokaryotic or eukaryotic virus promoters. In preferred embodiment the CMV promoter is used.

The promoter is typically located near the gene it regulates, on the same strand and upstream i.e. towards the 5′ region of the sense strand and it facilitates transcription.

According to an embodiment of the present invention, the cloning sites can be selected from but not limited to AatI, AatII, Acc113I, Acc16I, Acc65I, AccIII, AclNI, AseI, AsnI, Asp718I, BalI,

BamHI, BanI, BanII, BbeI, BbiII, BbsI, BbuI, Bbv16II, BclI, BcoI, BglI, BglII, BlnI, Bs h1365I, BsiEI, BsiHKAI, BsiI, BspEIBspHI, BstZI, CciNI, Cfr10I, Cfr42I, Cfr9I, CfrI, Csp45I, DrdI, DsaIEaeI, EagI, Eam1104I, Eco47III, Eco52I, Eco57I, Eco88I, Eco91I, EcoICRI, Eco O109I, EcoRI, EcoT14I, FriOI, FseI, FspI, HaeII, Hin1I, HincII, HindII, KasI, Ksp632I, KspI, KpnI, LspI, MamI, MflI, MluNI, MroI, MroNI, MspA1I, NaeI, NarI, NcoI, NdeI, NgoAIV, Nh eI, NotI, NspBII, NspI, NspV, PacI, PaeI, PflMI, PinAI, Ple19I, Pme55I, PmeI, Ppu10I, PpuMI, PshBI, Psp124BI, Psp51I, PspAI, PspALI, PspEI, PstI, PstNHI, PvuI, PvuII, SacI, SacII, ScaI, S fcI, SfiI, SpeI, SphI, Van91I, VneI, XhoI, XhoII, XmaI, XmaIII, Zsp2I.

According to an embodiment of the present invention the translation terminator is selected from the group consisting of bovine growth hormone, adenovirus and Eukaryotic Virus translation terminator sequences. In a preferred embodiment, the translation terminator is BGH Poly A.

In a further embodiment, the internal ribosomal binding sites are selected from Picornavirus IRES, Aphthovirus IRES, Hepatitis A IRES, Hepatitis C IRES, Pestivirus IRES, Encephalomyocarditis virus IRES preferably Encephalomyocarditis virus IRES.

According to an embodiment of the present invention the expression enhancing element such as Matrix attachment region is selected from Chicken Lysozyme MAR, Drosophila Scs boundary element, hspSAP. MAR, Mouse T cell receptor TCRa, rat locus control region, β-globulin MAR, and apolipoprotein B SAR element.

In preferred embodiment the Chicken Lysozyme MAR (Sequence id 5) is cloned at 5′ flanking sequence or 3′ flanking sequence. In most preferred embodiment the Chicken Lysozyme MAR is cloned at both 5′ and 3′ flanking sequence of the transcriptional assembly.

In the present invention the gene of interest is cloned in the expression vector according to method known in the art (SAMBROOK, J.; FRITSCH, E. F. and MANIATIS, T. Molecular Cloning: a laboratory manual. 2^{nd} ed. N.Y., Cold Spring Harbor Laboratory, Cold Spring Harbor Laboratory Press, 1989. 1659). According to an embodiment of the present invention the gene of interest may encode suitable proteins and peptides and functional analogues thereof selected from tissue plasminogen activator, TNK-TPA, Darbepoietin, Erythropoietin, Insulin, GCSF, Interleukin, Tumor necrosis factor, Interferon, TNFR-IgGFc, Monoclonal antibodies such as Rituximab, Bevacizumab, Adalimumab, Trastuzumab (generic names) and their fragments like Fc region, Fab, GLP-I, GLP-II, IGF-I, IGF-II, Platelet derived growth factor; FVII, FVIII, FIV and FXIII, exendin-3, exendin 4, transcription factors like MYT-2, NF-κB repressing factor NRF, AML1/RUNX1, Gtx homeodomain protein, translation factors like Eukaryotic initiation factor 4G (eIF4G)a, Eukaryotic initiation factor 4Gl (elF4Gl)a, Death associated protein 5 (DAP5), oncogene like c-myc, L-myc, Pim-1, Protein kinase p58PITSLRE, p53 hormones such as gonadotropic hormones selected from Follicle stimulating hormone, Human Chorionic Gonadotropin, Human Leutinizing Hormone, etc. and immunoglobulin heavy chain binding protein (BiP), Heat shock protein 70, β-subunit of mitochondrial H+-ATP synthase, Ornithine decarboxylase, connexins 32 and 43, HIF-1a, APC Accordingly, functional analogues means proteins or peptides having similar or identical functional to their native proteins and peptides.

According to the invention, expression vector construct comprises an expression assembly further comprising an operably linked promoter, cloning sites, gene of interest, transcription terminator, intron, suitable antibiotic marker, TPL, VA gene I and II. This expression vector is referred as pZRCII which is disclosed in sequence id no. 4(6). In another embodiment the gene sequence of expression enhancing elements like Matrix attachment region (MAR) or SARs preferably MAR is further cloned in pZRCII. This new expression vector is referred as pZRCIII disclosed in sequence id no 6. A matrix attachment region is cloned at 5′ or 3′ flanking region of pZRCIII. In preferred embodiment matrix attachment regions is cloned at both 5′ and 3′ flanking region of pZRCIII. pZRCIII construct of the present invention is an advance over the vector known in prior art and enhance the expression of gene of interest significantly as well as improves transfection efficiencies. The present vector construct pZRCIII is suitable for expression of all proteins and peptides.

The suitable antibiotic marker in expression vector pZRCIII is selected from kanamycine, hygromycin puromycin and DHFR. In another embodiment the expression vector pZRCIII optionally carries the gene sequence of DHFR and/or internal ribosomal binding site (IRES).

In one embodiment, the present invention provides the novel expression vector construct for the expression of therapeutic proteins and peptides where the expression vector construct comprises the promoter operably linked to cloning sites, gene of interest, translation terminator, TPL (Seq ID no. 2), VA I and II genes (Seq ID no. 3), suitable antibiotic marker in combination with expression enhancing elements selected from MARs and/or SARs

In another embodiment, the present invention provides the novel expression vector construct for the expression of therapeutic proteins and peptides where the expression vector construct comprises the promoter operably linked to cloning sites, gene of interest, transcription terminator, intron (Seq ID no. 1), suitable marker and optionally internal ribosomal binding site in combination with expression enhancing elements selected from MARs and/or SARs.

The expression vector for the production of the desired expression of proteins and peptide comprises suitable elements but is not limited to the incorporation of 3 elements namely, Adenoviral Tripartite leader sequence at the 3′ end of promoter, a hybrid (chimeric) intron comprising of 5′ donor site of the adenovirus major late transcript and the 3′ splice site of mouse immunoglobulin which is placed at the 3′ end of the TPL (Seq ID no. 2), the adenoviral VA RNA I and II genes (Seq ID no. 3). The matrix attachment region is cloned at 5′ flanking sequence at Mlu I or 3′ flanking ‘sequence at the’ end of BGH Poly A sequence. The orientation of matrix attachment region is optional. Moreover MARs element of the present invention not only enhance the expression of desired gene synergistically in combination with Adenoviral Tripartite leader sequence, hybrid (chimeric) intron, TPL (Seq ID no. 2) and the adenoviral VA RNA I and II genes (Seq ID no. 3) but also increase the transfection efficiency and numbers of desired clone.

In a preferred embodiment, the present invention provides the novel expression vector construct for the expression of therapeutic proteins and peptides where the expression vector construct comprises the promoter operably linked to cloning sites, gene of interest, transcription terminator, TPL (Seq ID no. 2), VA I and II genes (Seq ID no. 3), Intron (Seq ID no. 1), suitable marker and optionally internal ribosomal binding in combination with expression enhancing elements selected from MARs and/or SARs. In embodiment, the expression vector comprises a Promoter or variant thereof, operably linked to gene of interest, VA I and II gene or variant thereof, TPL or variant thereof, Chimeric Intron or variant thereof, Antibiotic marker, Matrix attachment regions, Optionally internal ribosomal binding site, Bovine growth harmone polyadenylation

In one embodiment PZRCIII-gene of interest-Hygromycin vector contains cLysMARs setforth the in sequence id no 5 operably linked with gene of interest driven by a CMV promoter, TPL, a chimeric intron setforth in sequence id no 1, VA genes I and II setforth in sequence id no. 3, BGH polyadenylation and multiple cloning sites. Multiple cloning sites includes restriction sites like XhoI, NotI: Any gene of interest can be cloned at multiple cloning site like the chemically synthesised gene of the fusion protein Etanercept (TNFR-Fc) cloned into multiple cloning site of the vector having a chicken lysozyme MAR element both in the upstream and downstream of the expression cassette in combination with other regulatory elements such as a CMV promoter, TPL, a chimeric intron in the expression cassette and VA genes placed outside the expression cassette. The vector has a hygromycin resistance gene for selection of transfectants. As an example of a vector construct prepared according to this aspect of the present invention, the pZRCIII-etanerceptetanercept-Hygromycin vector is deposited under Budapest treaty and accession number is MTCC 5656.

In another embodiment PZRCIII-gene of interest-Neomycine vector contains C-Lys-MARs setforth the in sequence id no 5 operable linked with CMV promoter, TPL, A chimeric intron setforth in sequence id no 1, VA genes I and II setforth in sequence id no. 3, BGH polyadenylation and multiple cloning sites. Multiple cloning site includes restriction sites like XhoI, NotI. Any gene of interest can be cloned at multiple cloning site like the chemically synthesised gene of the fusion protein Etanercept (TNFR-Fc) cloned into multiple cloning site of the vector having a chicken lysozyme MAR element both in the upstream and downstream of the expression cassette in combination with other regulatory elements such as a CMV promoter, TPL, a chimeric intron in the expression cassette and VA genes placed outside the expression cassette. The vector has a hygromycin (neomycin) resistance gene for selection of transfectants.

As an example of a vector construct prepared according to this aspect of the present invention, the pZRCIII-etanercept-Neomycin vector is deposited under Budapest treaty and accession number is MTCC 5657.

In another embodiment PZRCIII-gene of interest-IRES-Hygromycin vector contains cLysMARs setforth the in sequence id no 5 operable linked with CMV promoter, TPL, a chimeric intron setforth in sequence id no 1, VA genes I and II setforth in sequence id no. 3, BGH polyadenylation and multiple cloning sites. Multiple cloning sites includes restriction sites like XhoI, NotI. Any gene of interest can be cloned at multiple cloning site like chemically synthesised genes of the FSH α and FSH β subunits cloned into multiple cloning site of the vector and both FSH α and FSH β subunits operably linked to each other by IRES, having a chicken lysozyme MAR element both in the upstream and downstream of the expression cassette in combination with other regulatory elements such as a CMV promoter, TPL, a chimeric intron in the expression casssete and VA genes placed outside the expression cassete. The vector has a hygromycin resistance gene for selection of transfectants. As an example of a vector construct prepared according to this aspect of the present invention, the pZRCIII FSH α-IRES-FSH β-hygromycin vector is deposited under Budapest treaty and accession number is MTCC 5655.

In an embodiment, the expression vector is transfected to mammalian host cell by processes known to a skilled person. The mammalian host cell may be selected from CHO (Chinese hamster ovary) cell line, BHK (Baby hamster kidney) cell line etc which are well known for commercial production of proteins. In another embodiment the transfected host cell is further transfected with a different vector containing suitable antibiotics selected from kanamycin, hygromycin, puromycin to increase further expression of gene of interest.

In one embodiment the transfected cell line is CHO K1 which is selected by using Hygromycin, puromycin, kanamycine, G418 or other antibiotics.

Furthermore the transfected cell lines are selected by using DHFR selection medium e.g. methotrexate, if the expression vector carries a genes of DHFR. This selection relies on a gradual increase in the selection pressure on the transfected cell-line. (Kaufman and Sharp, 1982; Schimke et al., 1982).

In yet another embodiment the transfected cell lines are selected in a Glutamine synthetase (GS) selection medium, e.g. methionine sulphoximine (MSX), as the expression vector carries a genes of Glutamine synthetase.

Hence the present invention provides a novel expression vector comprising a unique combination of regulatory elements which increase transcription and translation remarkably and also suppress the position effects of the gene integration, thus giving a synergistic effect to the stable, high expression of the recombinant protein. In addition it provides the production of therapeutic proteins and peptide, monoclonal antibodies at industrial scale in a time effective manner as the labour intensive screening of a huge number of clones is drastically reduced in presence of typical elements. In addition the present expression vector can be used for both transient as well as stable expression. The present invention is further illustrated with the help of examples. The examples are only for illustrative purpose and present invention is not limited to them only.

### Example 1

### Construction of pZRC III Vector

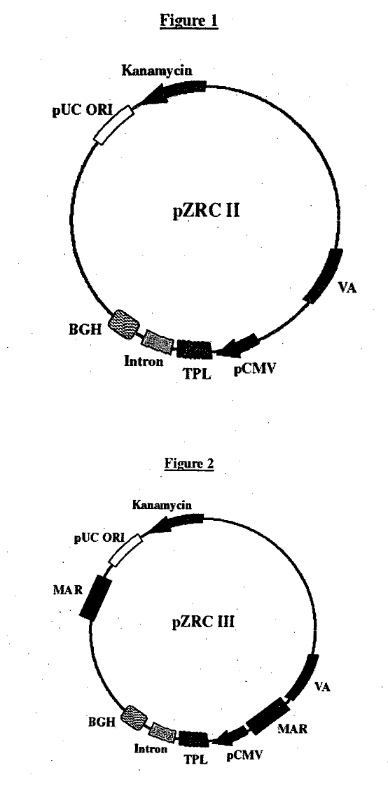

The whole transcription assembly with all the regulatory elements namely TPL, VA, CMV promoter, chimeric intron, and BGH polyadenylation and termination sequences, described in our earlier patent application WO2007017903, was chemically synthesized at GeneART, Germany. This whole assembly cloned in the cloning vector pMK (GeneART, Germany) was called pZRC II (FIG. 1, Seq ID No. 4,). Chicken lysozyme MAR DNA fragment (Seq ID No 5), (Phi-Van, L. and Stratling, W. H; Biochemistry 35 (33), 10735-10742 (1996)) was chemically synthesized and cloned in a cloning vector. Two chicken lysozyme MAR fragments were inserted as flanks on either side of the expression cassette in the pZRC II vector using the SacI and MluI sites which were already pre-designed into the vector. SacI overhang was added to the Chicken lysozyme MAR fragment by PCR using primers having SacI site. Specifically 40 cycles of PCR amplification were carried out using 100 picomoles of gene specific oligonucleotide primers in a volume of 50 μl containing 50 mM Tris-Cl (pH8.3), 2.5 mM MgCl₂, 250 μM each of the 4 dNTPs and 5 units of Pfu Polymerase. Each PCR amplification cycle consisted of incubations at 95° C. for 30 sec (denaturation), 62° C. for 30 sec (annealing) and 72° C. for 2 min (extension). Amplified product of the PCR reaction was resolved on a 1% Agarose gel. The desired fragment of approx 1664 base pairs size was excised out from the gel and purified using Qiagen Gel extraction kit. This purified DNA fragment was ligated into pZRC II vector after restriction digestion of both the vector and the purified PCR product with Sac I (MBI Fermentas, USA). The ligation product was transformed in E. coli Top 10F′ and transformants obtained were scored on the basis of kanamycin resistance. Plasmid DNA isolated from few such colonies was analyzed for the presence of Chicken lysozyme MAR fragment by restriction digestion using various restriction enzymes. One such plasmid found to be having the correct integration in pZRC II vector was named pZRC II 1MAR (Sac) intermediate vector.

To add Mlu I overhang to another Chicken lysozyme MAR fragment, it was subjected to 40 cycles of PCR amplification using 100 picomoles of gene specific oligonucleotide primers in a volume of 50 μl containing 50 mM Tris-Cl (pH8.3), 2.5 mM MgCl₂, 250 μM each of the 4 dNTPs and 5 units of Pfu Polymerase. Each PCR amplification cycle consisted of incubations at 95° C. for 30 sec (denaturation), 60° C. for 30 sec (annealing) and 72° C. for 2 min (extension). Amplified product of the PCR reaction was resolved on a 1% Agarose gel. The desired fragment of approx 1650 base pairs in size was excised out from the gel and purified using Qiagen Gel extraction kit. This purified DNA fragment was ligated into pZRC II-1MAR(Sac) vector after restriction digestion of both the vector and the purified PCR product with Mlu I (MBI Fermentas, USA). The ligation product was transformed in E. coli Top 10F′ and transformants were scored on the basis of kanamycin resistance. Plasmid DNA isolated from about 10 such colonies was analyzed for the presence of Chicken lysozyme MAR fragment at the MluI position by restriction digestion using various restriction enzymes. One such plasmid found to be having the correct integration of the cLysMAR in MluI site in pZRC II-1MAR(Sac) vector was named pZRC III (FIG. 2, Seq ID no 6).

### Example 2

### Construction of pZRC III-TNK Vector

Tenecteplase (TNKase or TNK-TPA) gene (Seq ID No 7) was chemically synthesized and cloned into a cloning vector pMK (Geneart, Germany). To clone TNK gene in the pZRC II vector, first EcoR I and Not I overhangs were incorporated into the TNK gene using 40 cycles of PCR amplification using 100 picomoles of specific oligonucleotide primers containing the above restriction sites in a volume of 50 μl containing 50 mM Tris-Cl (pH8.3), 2.5 mM MgCl₂, 250 μM each of the 4 dNTPs and 5 units of Pfu Polymerase. Each PCR amplification cycle consisted of incubations at 95° C. for 30 sec (denaturation), 60° C. for 45 sec (annealing) and 72° C. for 2 min (extension). Amplified product of the PCR reaction was resolved on a 1% Agarose gel. The desired fragment of approx 1710 base pairs in size was excised out from the gel and purified using Qiagen Gel extraction kit. This purified DNA fragment of TNK was digested with EcoR I and Not I and ligated into pZRC III vector (described in Example 1) digested with EcoR I and Not I (MBI Fermentas, USA). The ligation product was transformed in E. coli Top 10F′ and transformants were scored on the basis of kanamycin resistance. Plasmid DNA isolated from about 10 such colonies was analyzed for the presence of TNK fragment by restriction digestion using various restriction enzymes. One such plasmid, having the TNK gene integrated in the pZRC III vector was named, pZRC III-TNK.

### Example 3

### Construction of pZRC 1H-TNK-Hyg Vector

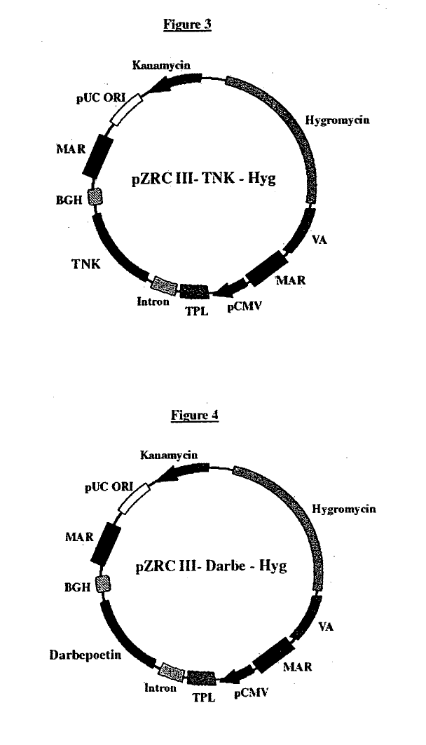

The Hygromycin transcription assembly of approx 1550 base pairs size and having the SV40 Promoter and terminator controlled Hygromycin resistance gene was blunt ended using Pfu polymerase (MBI Fermentas, USA) and then ligated into pZRC III-TNK vector, which was previously digested with Kpn I (MBI Fermentas, USA) and blunted using Pfu polymerase. The ligation product was transformed in E. coli Top 10F′ and transformants were scored on the basis of kanamycin resistance. Plasmid DNA isolated from few such colonies was analyzed for the presence of Hygromycin resistance gene by restriction digestion using various restriction enzymes. One such plasmid having the Hygromycin transcription assembly integrated in pZRC III-TNK vector was named, pZRC III-TNK-Hyg vector (FIG. 3, Seq ID No. 8). This vector was then subjected to DNA sequencing using automated DNA sequencer (ABI) to verify the sequence of the cloned TNK gene.

### Example 4

### Construction of pZRC III-Darbe-Hyg Vector

pZRC-EPO (WO2007017903) was used as a template for carrying out site directed mutagenesis of the erythropoietin gene to obtain Darbepoetin gene fragment (Seq ID No. 9 and the corresponding DNA sequence ID 21) of approx 600 bp which was then cloned in TA vector pTZ57R (MBI Fermentas) and called, pTZ57R-Darbe.

pZRC III-TNK-Hyg was digested with Xho I and Not I to remove the TNK gene and the remaining high molecular weight DNA was used as the vector for ligation with Darbepoetin gene insert. pTZ57R-Darbe was digested with Xho I and Not I to gel isolate approx. 600 bp Darbepoetin gene fragment. Ligation of both the vector and insert was carried out and the ligation product was transformed in E. coli Top 10F′ and transformants were scored on the basis of kanamycin resistance. Plasmid DNA isolated from few such colonies was analyzed for the presence of Darbepoetin gene by restriction digestion using various restriction enzymes. One such plasmid having the integrated Darbepoetin gene was named pZRC III-Darbe-Hyg vector (FIG. 4, Seq ID No. 10). This vector was then subjected to DNA sequencing using automated DNA sequencer (ABI) to verify the sequence of the cloned Darbepoetin gene.

### Example 5

### Construction of pZRC III-Etanercept-Hyg Vector

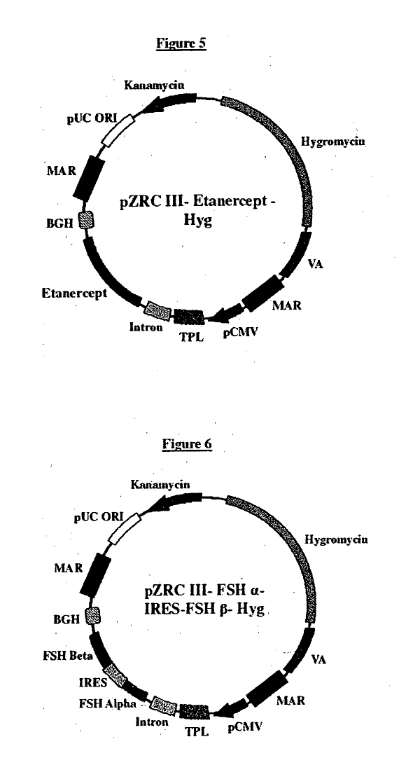

Vector pZRC III-Darbe-Hyg was digested with Xho I and Not I enzymes (MBI Fermentas) to remove the Darbepoetin gene of approx 600 bp and generate the vector backbone of approx 9430 bp. Chemically synthesized, CHO codon optimized, Etanercept gene (Seq ID No. 11 and corresponding DNA sequence ID 22) of approx. 1481 bp was isolated from the cloning vector using Xho I and Not I (MBI Fermentas) to obtain the insert. Ligation of both the vector and insert was carried out and the ligation product was transformed in E. coli Top 10F′ and transformants were scored on the basis of kanamycin resistance. Plasmid DNA isolated from few such colonies was analyzed for the presence of Etanercept gene by restriction digestion using various restriction enzymes. One such plasmid having the integrated Etanercept gene was named pZRC III-Etanercept-Hyg vector (FIG. 5, Seq ID No. 12). This vector was then subjected to DNA sequencing using automated DNA sequencer (ABI) to verify the sequence of the cloned Etanercept gene.

### Example 6

### Construction of pZRC III-FSH α-IRES-FSH β-Hyg Vector

a) Construction of pZRC III-FSH α-Hyg Vector

A vector backbone of approx. 9430 bp was generated by digesting pZRC III-Darbe-Hyg vector with Xho I and Not I (MBI Fermentas) to remove the approx. 600 bp of Darbepoetin gene. Chemically synthesized gene of FSH alpha subunit (Seq ID No. 13 the corresponding DNA sequence ID 23) of approx. 359 bp was isolated from the Geneart cloning vector pMA, using the enzymes Xho I and Not I (MBI Fermentas). Ligation of both the vector and insert was carried out and the ligation product was transformed in E. coli Top 10F′ and transformants were scored on the basis of kanamycin resistance. Plasmid DNA isolated from few such colonies was analyzed for the presence of FSH alpha subunit gene by restriction digestion using various restriction enzymes. One such plasmid having the integrated FSH alpha subunit gene was named, pZRC III-FSH α-Hyg vector.

b) Construction of pZRC III-FSH α-IRES-FSH β-Hyg Vector

Vector pZRC III-FSH α-Hyg was digested with Not I (MBI Fermentas) to generate the vector backbone of approx. 9790 bp. An IRES gene fragment of approx. 591 bp (Seq ID No. 14) was isolated from the vector pIRES Hyg using the enzymes Not I and Xma I (MBI Fermentas), to obtain the first insert. Chemically synthesized gene of FSH beta subunit (Seq ID No. 15 the corresponding DNA sequence ID 24) of approx. 401 bp was isolated from the Geneart cloning vector pMA using the enzymes Xma I and Not I (MBI Fermentas), to obtain the second insert. The 2 inserts were fused and the fused gene product was ligated with the vector above. The ligation product was then transformed in E. coli Top 10F′ and transformants were scored on the basis of kanamycin resistance. Plasmid DNA isolated from few such colonies was analyzed for the presence of IRES and FSH beta subunit genes by restriction digestion using various restriction enzymes. One such plasmid having the integrated IRES and FSH beta subunit genes was named pZRC III-FSH α-IRES-FSH β-Hyg vector (FIG. 6, Seq ID No. 16). This vector was then subjected to DNA sequencing using automated DNA sequencer (ABI) to verify the sequence of the cloned FSH α and FSH β genes. The sequence of the cloned genes was confirmed by using automated DNA sequencer (ABI).

### Example 7

### Construction of pZRC III-FSH β-IRES-FSH α-Hyg Vector

a) Construction of pZRC III-FSH β-Hyg vector

A vector pZRC III-FSH α-IRES-FSH β-Hyg was digested with Xho I and Not I (MBI Fermentas) to generate the vector backbone of approx. 9430 bp after removal of FSH α, IRES and FSH β genes. Chemically synthesized gene of FSH beta subunit of approx. 401 bp was isolated from the Geneart cloning vector pMA using the enzymes Xho I and Not I (MBI Fermentas). Ligation of both the vector and insert was carried out and the ligation product was transformed in E. coli Top 10F′ and transformants were scored on the basis of kanamycin resistance. Plasmid DNA isolated from few such colonies was analyzed for the presence of FSH beta subunit gene by restriction digestion using various restriction enzymes. One such plasmid having the integrated FSH beta subunit gene was named pZRC III-FSH β-Hyg vector.

b) Construction of pZRC III-FSH β-IRES-FSH α-Hyg Vector

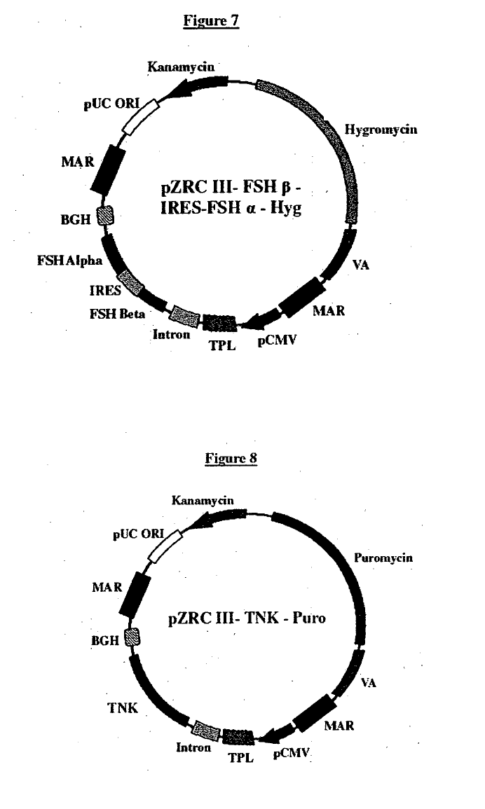

Vector pZRC III-FSH β-Hyg was digested with Not I (MBI Fermentas) to generate the vector backbone of approx. 9790 bp. An IRES gene fragment of approx. 591 bp was isolated from the vector pIRES Hyg using the enzymes Not I and Xho I (MBI Fermentas), to obtain the first insert (IRES). Chemically synthesized gene of FSH alpha subunit of approx 359 bp was isolated from the Geneart cloning vector pMA using the enzymes Xho I and Not I (MBI Fermentas), to obtain the second insert. This was followed by three piece ligation of the vector and the 2 inserts. The ligation product was then transformed in E. coli Top 10F′ and transformants were scored on the basis of kanamycin resistance. Plasmid DNA isolated from few such colonies was analyzed for the presence of IRES and FSH alpha subunit genes by restriction digestion using various restriction enzymes. One such plasmid having the integrated IRES and FSH alpha subunit genes was named pZRC III FSH β-IRES-FSH α-Hyg vector. (FIG. 7). The sequence of the cloned genes was confirmed by using automated DNA sequencer (ABI)

### Example 10

### Construction of pZRC III-TNK-Puromycin Vector

The Puromycin transcription assembly of approx. 1110 base pairs in size having the SV40 Promoter and terminator controlled Puromycin resistance gene and carrying BamHI compatible ends ligated into pZRC III-TNK vector which was also digested with BamH I (MBI Fermentas, USA). The ligation product was transformed in E. coli Top 10′ and transformants were scored on the basis of kanamycin resistance. Plasmid DNA isolated from few such colonies was analyzed for the presence of Puromycin fragment by restriction digestion using various restriction enzymes. One such plasmid having the Puromycin transcription assembly integrated in pZRC III-TNK vector was named pZRC III-TNK-Puro vector (FIG. 8, Seq ID No. 17). The sequence of the cloned TNK gene was confirmed by using automated DNA sequencer (ABI).

### Example 11

### Construction of pZRC Darbepoetin-Puro Vector

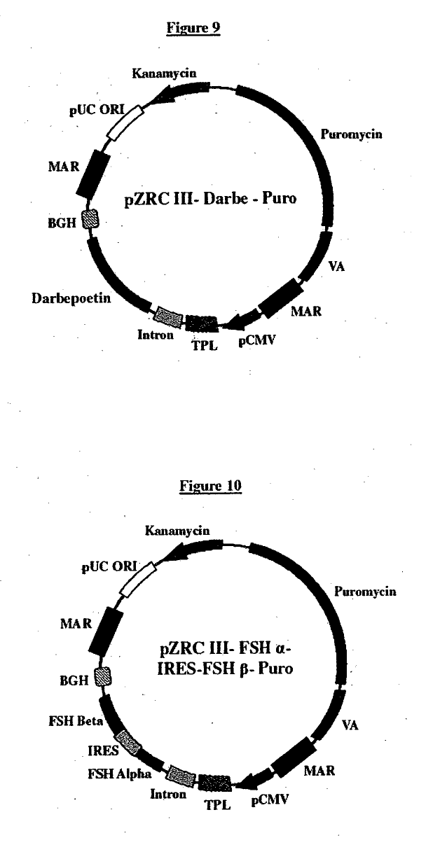

pZRC III-TNK-Puro was digested with Xho I and Not I (MBI Fermentas) to remove the TNK gene fragment. pTZ57R-Darbe was digested with Xho I and Not I to gel isolate approx. 600 bp Darbepoetin gene fragment. Ligation of both the vector and insert was done. The ligation product was transformed in E. coli Top 10F′ and transformants were scored on the basis of kanamycin resistance. Plasmid DNA isolated from few such colonies was analyzed for the presence of Darbepoetin gene fragment by restriction digestion using various restriction enzymes. One such plasmid was having the integrated Darbepoetin gene named pZRC III-Darbe-Puro vector (FIG. 9, Seq ID No. 18). The sequence of the cloned Darbepoietin gene was confirmed by using automated DNA sequencer (ABI).

### Example 12

### Construction of pZRC III-FSH α-IRES-FSH β-Puro Vector

Hygromycin cassette from pZRC III-FSH α-IRES-FSH β-Hyg vector was removed and replaced with the Puromycin transcription assembly of approx. 1110 base pairs in size having the SV40 Promoter and terminator controlled Puromycin resistance gene amplified from pZRC III-Darbe-Puro using a PCR reaction with specific oligonucleotide primers. The obtained PCR product was digested using specific endonucleases and used for further ligations with the vector backbone. The ligation product was transformed in E. coli Top 10F′ and transformants were scored on the basis of kanamycin resistance. Plasmid DNA isolated from few such colonies was analyzed for the presence of Puromycin resistance gene by restriction digestion using various restriction enzymes. One such plasmid having the integrated Puromycin resistant gene was named pZRC III-FSH α-IRES-FSH β-Puro vector (FIG. 10)

### Example 13

### Construction of pZRC III-FSH α-IRES-FSH β-Neo Vector

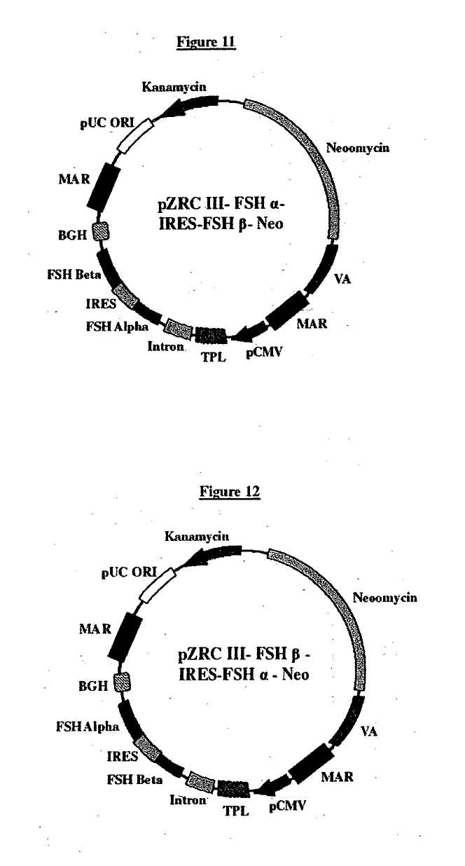

Vector pZRC III-FSH α-IRES-FSH β-Puro was digested with Pac I and Bam HI (MBI Fermentas) to generate the vector backbone of approx. 9980 bp after removal of Puromycin resistant gene. Neomycin resistant gene of approx. 1518 bp was isolated from pcDNA 3.1 (Invitrogen) plasmid. Ligation of both the vector and insert was carried out and the ligation product was transformed in E. coli Top 10F′ and transformants were scored on the basis of kanamycin resistance. Plasmid DNA isolated from few such colonies was analyzed for the presence of Neomycin resistant gene by restriction digestion using various restriction enzymes. One such plasmid having the integrated Neomycin resistant subunit gene was named pZRC III-FSH α-IRES-FSH β-Neo vector (FIG. 11). The sequence of FSH α, IRES and FSH β genes was confirmed by using automated DNA sequencer (ABI).

### Example 14

### Construction of pZRC III-FSH β-IRES-FSH α-Neo Vector

a) Construction of pZRC III-FSH β-Neo Vector

Vector pZRC III-FSH α-IRES-FSH β-Neo was digested with Xho I and Not I (MBI Fermentas) to generate the vector backbone of approx. 9400 bp after removal of FSH α, IRES and FSH β genes. Chemically synthesized gene of FSH beta subunit of approx. 401 bp was isolated from the Geneart cloning vector pMA using the enzymes Xho I and Not I (MBI Fermentas). Ligation of both the vector and insert was carried out and the ligation product was transformed in E. coli Top 10F′ and transformants were scored on the basis of kanamycin resistance. Plasmid DNA isolated from few such colonies was analyzed for the presence of FSH beta subunit gene by restriction digestion using various restriction enzymes. One such plasmid having the integrated FSH beta subunit gene was named pZRC III-FSH β-Neo vector.

b) Construction of pZRC III FSH β-IRES-FSH α-Neo Vector
Vector pZRC III-FSH β-Neo was digested with Not I (MBI Fermentas) to generate the vector backbone of approx. 9800 bp. The IRES DNAfragment of approx. 591 bp was isolated from the vector pIRES Hyg using the enzymes Not I and Xho I (MBI Fermentas), to obtain the first insert. Chemically synthesized gene of FSH alpha subunit of approx. 359 bp was isolated from the Geneart vector pMA using the enzymes Xho I and Not I (MBI Fermentas), to obtain the second insert. This was followed by three piece ligation of the vector and the 2 inserts. The ligation product was then transformed in E. coli Top 10F′ and transformants were scored on the basis of kanamycin resistance. Plasmid DNA isolated from few such colonies was analyzed for the presence of IRES and FSH alpha subunit genes by restriction digestion using various restriction enzymes. One such plasmid having the integrated IRES and FSH alpha subunit genes was named obtain pZRC III FSH β-IRES-FSH α-Neo vector (FIG. 12). The sequence of the cloned genes was confirmed by using automated DNA sequencer (ABI).

### Example 17

### Construction of pZRC III-Etanercept-Neo Vector

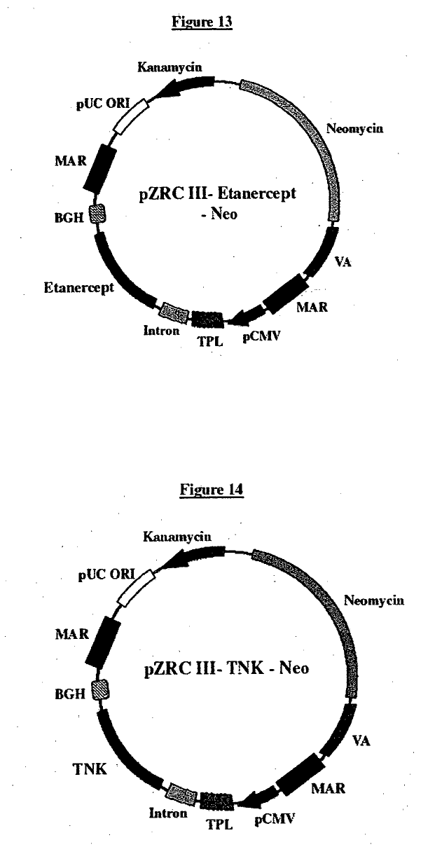

Vector pZRC III-FSH α-IRES-FSH β-Neo was digested with Xho I and Not I (MBI Fermentas) enzymes to remove the FSH α, IRES and FSH β genes, and obtain the vector backbone of approx. 9400 bp to be used for cloning the Etanercept gene. The approx. 1481 bp gene of Etanercept was isolated from the vector pZRC III-Etanercept-Hyg using Xho I and Not I (MBI Fermentas) enzymes to obtain the insert. Ligation of both the vector and insert was carried out and the ligation product was transformed in E. coli Top 10F′ and transformants were scored on the basis of kanamycin resistance. Plasmid DNA isolated from about few colonies was analyzed for the presence of Etanercept gene by restriction digestion using various restriction enzymes. One such plasmid having the integrated Etanercept gene was named pZRC III-Etanercept-Neo vector (FIG. 13, Seq ID No. 19). The sequence of the cloned Etanercept gene was confirmed by using automated DNA sequencer (ABI).

### Example 18

### Construction of pZRC III-TNK-Neo Vector

Vector pZRC III-Etanercept-Neo was digested with the enzymes Xho I and Not I (MBI Fermentas) to remove the approx. 1481 Etanercept gene and obtain the vector construct of approx. 9400 bp. The insert of TNK gene of approx. 1692 bp was obtained after digesting the vector pZRC III-TNK-Hyg with Xho I and Not I enzymes (MBI Fermentas). Ligation of both the vector and insert was carried out and the ligation product was transformed in E. coli Top 10F′ and transformants were scored on the basis of kanamycin resistance. Plasmid DNA isolated from few such colonies was analyzed for the presence of TNK gene by restriction digestion using various restriction enzymes. One such plasmid having the integrated TNK gene was named pZRC III-TNK-Neo vector (FIG. 14,). The sequence of the cloned TNK gene was confirmed by using automated DNA sequencer (ABI).

### Example 19

### Expression of Etanercept

Set I—Stable Transfections in CHO-K1-S Cell Line Using pZRC III-Etanercept-Hyg Vector

Freestyle™ CHO-K1-S cell were cultivated routinely in PowerCHO2 CD medium (chemically defined medium, Lonza) supplemented with 4 mM Glutamine. Cells were maintained under agitation (120 rpm) at 37° C., and 5% CO2 in a humidified incubator. Cells were counted every 3rd/4th day and given a complete medium exchange. Transfections were carried out using Neon Transfection system (Invitrogen). One day prior to transfection, CHO-KI-S cells were passaged into fresh medium and allowed at least one doubling before use for transfection. Transfections were carried out using Sgs I (Asc I) linearised pZRC III-Etanercept-Hyg plasmid as per standard protocols described by the manufacturer (Invitrogen). After Transfection, the cells were transferred into one well of a 24 well plate, containing 1 mL of pre-warmed culture medium. Cells were maintained at 37° C., 5% CO2 in a humidified incubator. On the next day, for minipool generation, transfected population was plated in 96 well plates in Pro CHO 5 medium (Lonza) supplemented with 4 mM Glutamine and 600 μg/ml of Hygromycin. After 15-30 days, supernatants from 96 well plates were removed for product formation analysis by ELISA. The selected high expressing minipools were then transferred to 24 well plate and subsequently to 6 well plate in PowerCHO2 CD medium (chemically defined medium, Lonza) supplemented with 4 mM Glutamine and 600 μg/ml of Hygromycin and expression levels were analyzed at each level by ELISA. High expressing minipools were chosen to carry out single cell dimiting dilution in 96 well plates in Pro CHO 5 medium (Lonza) supplemented with 4 mM Glutamine and 600 μg/ml of Hygromycin. After around 15-30 days, supernatants from 96 well plates were removed for product formation analysis by ELISA. The selected high expressing clones were then transferred to 24 well plate and then to 6 well plate in PowerCHO2 CD medium (chemically defined medium, Lonza) supplemented with 4 mM Glutamine and 600 μg/ml of Hygromycin and expression levels were analyzed at each level by ELISA. High producing clones were selected for re transfections.

Set II—Stable Retransfections of Clones Obtained from Set I Using pZRC III-Etanercept-Neo Vector

High expressing clones were chosen to carry out re-transfections using pZRC III-Etanercept-Neo plasmid linearized by Sgs I (Acs I) by the same procedure as in Set I transfections. On the next day, for minipool generation, transfected population was plated in 96 well plates in Pro CHO 5 medium (Lonza) supplemented with 4 mM Glutamine, 600 μg/ml of Hygromycin, and 500 μg/ml of Neomycin. After 15-30 days, supernatants from 96 well plates were removed for product formation analysis by ELISA. The selected high expressing minipools were then transferred to 24 well plate and then to 6 well plate in PowerCHO2 CD medium (chemically defined medium, Lonza) supplemented with 4 mM Glutamine and said antibiotic pressures and expression levels were analyzed at each level by ELISA. High producing minipools were chosen to carry out single cell limiting dilution in 96 well plates in Pro CHO 5 medium (Lonza) supplemented with 4 mM Glutamine. Again after 15-30 days, supernatants from 96 well plates were removed for product formation analysis by ELISA. The selected high expressing clones were then transferred to 24 well plate and then to 6 well plate in PowerCHO2 CD medium (chemically defined medium, Lonza) supplemented with 4 mM Glutamine and expression levels were analyzed at each level by ELISA. High expressing retransfected clones were selected to analyse the product formation in shake tubes in fed batch mode. These experiments were carried out using these selected clones in spin tubes on shaker (Kuhner-Germany) at 120 rpm, 37° C., 5% CO₂. Clones yielded production levels in the range of approx. 700 mg/l to 1000 mg/l in 12 days.

### Example 20

### Expression of TNK

Set I—Stable Transfections in CHO-K1-S Cell Line Using pZRC III-TNK-Hyg Vector

Freestyle™, CHO-K 1-S cell were cultivated routinely in PowerCHO2 CD medium (chemically defined medium, Lonza) supplemented with 4 mM Glutamine. Cells were maintained under agitation (120 rpm) at 37° C., and 5% CO2 in a humidified incubator. Cells were counted every 3rd/4th day and given a complete medium exchange. Transfections were carried out using Neon Transfection system (Invitrogen). One day prior to transfection, CHO-KI-S cells were passaged into fresh medium and allowed at least one doubling before use for transfection. Transfections were carried out using Sgs I (Asc I) linearised pZRC III-TNK-Hyg plasmid as per standard protocols described by the manufacturer (Invitrogen). After Transfection, the cells were transferred into one well of a 24 well plate, containing 1 mL of pre-warmed culture medium. Cells were maintained at 37° C., 5% CO2 in a humidified incubator. On the next day, for minipool generation, transfected population was plated in 96 well plates in PowerCHO2 CD medium (chemically defined medium, Lonza) supplemented with 4 mM Glutamine and 500 μg/ml of Hygromycin. After 15-30 days, supernatants from 96 well plates were removed for product formation analysis by ELISA. The selected high expressing minipools were then transferred to 24 well plate and subsequently to 6 well plate and expression levels were analyzed at each level by ELISA. High expressing minipools were chosen to carry out single cell dimiting dilution in 96 well plates in PowerCHO2 CD medium (chemically defined medium, Lonza) supplemented with 4 mM Glutamine and 500 μg/ml of Hygromycin. After around 15-30 days, supernatants from 96 well plates were removed for product formation analysis by ELISA. The selected high expressing clones were then transferred to 24 well plate and then to 6 well plate and expression levels were analyzed at each level by ELISA. High producing clones were selected for re transfections. High expressing clones were selected to analyse the product formation in shake tubes in fed batch mode. These experiments were carried out using these selected clones in 10 ml media in spin tubes on shaker (Kuhner-Germany) at 230 rpm, 37° C., 5% CO₂. Clone yielded productions levels of 150 mg/l in 9 days.

Set IIa—Stable Re-Transfections of Clones Obtained from Set I Using pZRC III-TNK-Puro Vector

High expressing clones were chosen to carry out re-transfections using pZRC III-TNK-Puro plasmid linerised by Sgs I (Acs I) by the same procedure as in Set I transfections. On the next day, for minipool generation, transfected population was plated in 96 well plates in PowerCHO2 CD medium (chemically defined medium, Lonza) supplemented with 4 mM Glutamine, 500 μg/ml of Hygromycin, and 3 ug/ml of Puromycin. After 15-30 days, supernatants from 96 well plates were removed for product formation analysis by ELISA. The selected high expressing minipools were then transferred to 24 well plate and then to 6 well plate and expression levels were analysed at each level by ELISA. High producing minipools were chosen to carry out single cell limiting dilution in 96 well plates in PowerCHO2 CD medium (chemically defined medium, Lonza) supplemented with 4 mM Glutamine. Again after 15-20 days, supernatants from 96 well plates were removed for product formation analysis by ELISA. The selected high expressing clones were then transferred to 24 well plate and then to 6 well plate and expression levels were analysed at each level by ELISA. High expressing clones were selected to analyse the product formation in shake tubes in fed batch mode. These experiments were carried out using these selected clones in spin tubes on shaker (Kuhner-Germany) at 120 rpm, 37° C., 5% CO₂. Clones yielded productions, levels of 290 mg/l in 11 days.

Set IIb—Stable Re-Transfections of Clones Obtained from Set I Using pZRC III-TNK-Neo Vector

High producing clones were chosen to carry out re-transfections using pZRC III-TNK-Neo plasmid linearized by Sgs I (Acs I) by the same procedure as in Set I transfections. On the next day, for minipool generation, transfected population was plated in 96 well plates in Pro CHO 5 medium (Lonza) supplemented with 4 mM Glutamine, 600 μg/ml of Hygromycin, and 500 μg/ml of Neomycin. After 15-30 days, supernatants from 96 well plates were removed for product formation analysis by ELISA. The selected high expressing minipools were then transferred to 24 well plate and then to 6 well plate in PowerCHO2 CD medium (chemically defined medium, Lonza) supplemented with 4 mM Glutamine and the mentioned antibiotic pressures and expression levels were analyzed at each level by ELISA. High producing minipools were chosen to carry out single cell limiting dilution in 96 well plates in Pro CHO 5 medium (Lonza) supplemented with 4 mM Glutamine. Again after 15-30 days, supernatants from 96 well plates were removed for product formation analysis by ELISA. The selected high expressing clones were then transferred to 24 well plate and then to 6 well plate in PowerCHO2 CD medium (chemically defined medium, Lonza) supplemented with 4 mM Glutamine and expression levels were analyzed at each level by ELISA. High expressing clones were selected to analyze the product formation in shake tubes in fed batch mode. These experiments were carried out using these selected clones in spin tubes on shaker (Kuhner-Germany) at 120 rpm, 37° C., 5% CO₂. Clone yielded productions levels of 390 mg/l in 10 days.

### Example 21

### Expression of Darbepoetin Using pZRC III-Darbe-Hyg Vector

Set I—Stable Transfections in CHO-K1-S Cell Line Using pZRC III-Darbe-Hyg Vector

Freestyle™ CHO-K 1-S cell were cultivated routinely in PowerCHO2 CD medium (chemically defined medium, Lonza) supplemented with 4 mM Glutamine. Cells were maintained under agitation (120 rpm) at 37° C., and 5% CO2 in a humidified incubator. Cells were counted every 3rd/4th day and given a complete medium exchange. Transfections were carried out using Neon Transfection system (Invitrogen). One day prior to transfection, CHO-KI-S cells were passaged into fresh medium and allowed at least one doubling before use for transfection. Transfections were carried out using Sgs I (Asc I) linearised pZRC III-Darbe-Hyg plasmid as per standard protocols described by the manufacturer (Invitrogen). After transfection of DNA, the cells were transferred into one well of a 24 well plate, containing 1 mL, of pre-warmed culture medium. Cells were maintained at 37° C., 5% CO2 in a humidified incubator. On the next day, for minipool generation, transfected population was plated in 96 well plates in PowerCHO2 CD medium (chemically defined medium, Lonza) supplemented with 4 mM Glutamine and 600 μg/ml of Hygromycin. After 15-20 days, supernatants from 96 well plates were removed for product formation analysis by ELISA. The selected high expressing minipools were then transferred to 24 well plate and then to 6 well plate and expression levels were analyzed at each level by ELISA. Two high expressing minipools were chosen to carry out single cell limiting dilution in 96 well plates in ProCHO5 medium (Lonza) supplemented with 4 mM Glutamine and 600 μg/ml of Hygromycin. Again after 15-30 days, supernatants from 96 well plates were removed for product formation analysis by ELISA. The selected high expressing clones were then transferred to 24 well plate and then to 6 well plate and expression levels were analyzed at each level by ELISA. High expressing clones were selected to analyse the product formation in shake tubes in fed batch mode. These experiments were carried out using these selected clones in 10 ml media in spin tubes on shaker (Kuhner-Germany) at 230 rpm, 37° C., 5% CO2. Clone yielded productions levels of 340 mg/l in 11 days.

### Example 22

### Expression of FSH

Set I—Stable Transfections in CHO-K1-S Cell Line Using pZRC III-FSH α-IRES-FSH β-Hyg Vector

Freestyle™ CHO-K1-S cell were cultivated routinely in PowerCHO2 CD medium (chemically defined medium, Lonza) supplemented with 4 mM Glutamine. Cells were maintained under agitation (120 rpm) at 37° C., and 5% CO2 in a humidified incubator. Cells were counted every 3rd/4th day and given a complete medium exchange. Transfections were carried out using Neon Transfection system (Invitrogen). One day prior to transfection, CHO-KI-S cells were passaged into fresh medium and allowed at least one doubling before use for transfection. Transfections were carried out using Sgs I (Asc I) linearised pZRC III-FSH α-IRES-FSH β-Hyg plasmid as per standard protocols described by the manufacturer (Invitrogen). After Transfection, the cells were transferred into one well of a 24 well plate, containing 1 mL of pre-warmed culture medium. Cells were maintained at 37° C., 5% CO2 in a humidified incubator. On the next day, for minipool generation, transfected population was plated in 96 well plates in Pro CHO 5 medium (Lonza) supplemented with 4 mM Glutamine and 600 μg/ml of Hygromycin. After 15-30 days, supernatants from 96 well plates were removed for product formation analysis by ELISA. The selected high expressing minipools were then transferred to 24 well plate and subsequently to 6 well plate in PowerCHO2 CD medium (chemically defined medium, Lonza) supplemented with 4 mM Glutamine and said antibiotic pressure and expression levels were analyzed at each level by ELISA. High expressing minipools were chosen to carry out single cell limiting dilution in 96 well plates in Pro CHO 5 medium (Lonza) supplemented with 4 mM Glutamine and 600 μg/ml of Hygromycin. After around 15-30 days, supernatants from 96 well plates were removed for product formation analysis by ELISA. The selected high expressing clones were then transferred to 24 well plate and then to 6 well plate in PowerCHO2 CD medium (chemically defined medium, Lonza) supplemented with 4 mM Glutamine and said antibiotic pressure and expression levels were analyzed at each level by ELISA. High producing clones were selected for retransfections.

Set II—Stable Re-Transfections of Clones Obtained from Set I Using pZRC III-FSH α-IRES-FSH β-Neo Vector

Clones were chosen to carry out re-transfections using pZRC III-FSH α-IRES-FSH β-Neo plasmid linearized by Sgs I (Acs I) by the same procedure as Set I transfections. On the next day, for minipool generation, transfected population was plated in 96 well plates in Pro CHO 5 medium (Lonza) supplemented with 4 mM Glutamine, 600 μg/ml of Hygromycin, and 500 ug/ml of Neomycin. After 15-30 days, supernatants from 96 well plates were removed for product formation analysis by ELISA. The selected high expressing minipools were then transferred to 24 well plate and then to 6 well plate in PowerCHO2 CD medium (chemically defined medium, Lonza) supplemented with 4 mM Glutamine and said antibiotic pressures and expression levels were analyzed at each level by ELISA High producing retransfected minipools were chosen to carry out single cell limiting dilution in 96 well plates in Pro CHO 5 medium (Lonza) supplemented with 4 mM Glutamine and mentioned antibiotic pressure. Again after 15-30 days, supernatants from 96 well plates were removed for product formation analysis by ELISA. The selected high expressing clones were then transferred to 24 well plate and then to 6 well plate in PowerCHO2 CD medium (chemically defined medium, Lonza) supplemented with 4 mM Glutamine and mentioned antibiotic pressure and expression levels were analyzed at each level by ELISA. High expressing clones namely were selected to analyse the product formation in shake tubes in fed batch mode. These experiments were carried out using these selected clones in spin tubes on shaker (Kuhner-Germany) at 120 rpm, 37° C., 5% CO₂. Production levels were obtained in range of approx. 20 mg/l to 50 mg/l in 10 days with different clones.

1. An expression vector comprising a promoter operably linked to the gene of interest, expression enhancement elements, TPL, VA I and II genes or variants thereof, translation terminator and an antibiotic marker wherein the expression enhancement element is a chromatin attachment region.

2. The expression vector as claimed in claim 1, which further comprises
a) Intron or variants thereof; and
b) optionally an Internal ribosomal binding site or variants thereof.

3. The expression vector as claimed in claim 1, wherein the chromatin attachment region is a suitable matrix attachment region.

4. The expression vector as claimed in claim 1, wherein the chromatin attachment region is a suitable scaffold attachment region.

5. The expression vector as claimed in claim 3, wherein the matrix attachment region is selected from Drosophila Scs boundary element, hspSAP MAR, cLysMARs, Mouse T cell receptor TCRα, Rat locus control region, and β-globin MAR.

6. The expression vector as claimed in claim 3, wherein the matrix attachment region is cLysMARs.

7. The expression vector as claimed in claim 5, wherein the cLysMARs has nucleotide sequence set forth in SEQ ID NO:5.

8. The expression vector as claimed in claim 1, wherein the promoter is selected from the group consisting of CMV promoter, SV40 promoter, adenovirus promoter, Beta actin promoter, metallothionin Promoters or other prokaryotic or eukaryotic virus promoters.

9. The expression vector as claimed in claim 2, wherein the internal ribosomal binding site is Encephalomyocarditis virus IRES.

10. The expression vector as claimed in claim 2, wherein the internal ribosomal binding site has nucleotide sequence set forth in SEQ ID NO:14.

11. The expression vector as claimed in claim 1, wherein the VA I and II genes have nucleotide sequence set forth in SEQ ID NO:3.

12. The expression vector as claimed in claim 1, wherein the TPL has nucleotide sequence set forth in SEQ ID NO:2.

13. The expression vector as claimed in claim 2, wherein the chimeric Intron has nucleotide sequence set forth in SEQ ID NO:1.

14. The expression vector as claimed in claim 1, wherein the gene of interest encodes proteins and peptides and analogues thereof selected from tissue plasminogen activator, TNK-TPA, Darbepoietin, Erythropoietin, Insulin, GCSF, Interleukin, Tumor necrosis factor, Interferon, INFR-IgGFc, monoclonal antibodies selected from rituximab, bevacizumab, adalimumab, trastuzumab and their fragments like Fc region, Fab, GLP-I, GLP-II, IGF-I, IGF-II, Platelet derived growth factor, FVII, FVIII, FIV and FXIII, exendin-3, exendin 4, transcription factors like MYT-2, NF-κB repressing factor NRF, AML1/RUNX1, Gtx homeodomain protein, translation factors selected from Eukaryotic initiation factor 4G (elF4G)a, Eukaryotic initiation factor 4Gl (eIF4Gl)a, Death associated protein 5 (DAP5), oncogene like c-myc, L-myc, Pim-1, Protein kinase p58PITSLRE, p53 hormones selected from gonadotropic hormones selected from Follicle stimulating hormone, Human Chorionic Gonadotropin, Human Luteinizing Hormone, and immunoglobulin heavy chain binding protein (BiP), Heat shock protein 70, β-subunit of mitochondrial H+-ATP synthase, Ornithine decarboxylase, connexins 32 and 43, HIF-1a, and APC.

15. The expression vector as claimed in claim 1, wherein the antibiotic marker is selected from kanamycin, puromycin, hygromycin, and neomycin.

16. The expression vector as claimed in claim 1, wherein the cLysMARs is cloned at either flank of the expression cassette.

17. The expression vector as claimed in claim 1, which comprises a gene of interest operably linked to
a) a Promoter or variant thereof;
b) VA I and II gene or variant thereof;
c) TPL or variant thereof;
d) chimeric Intron or variant thereof;
e) antibiotic marker;
f) matrix attachment regions;
g) Optionally internal ribosomal binding site; and
h) Bovine growth harmone polyadenylation sequence.

18. The expression vector as claimed in claim 1, having accession number MTCC 5655.

19. The expression vector as claimed in claim 1, having accession number MTCC 5656.

20. The expression vector as claimed in claim 1, having accession number MTCC 5657

21. A host cell transformed with vector as claimed in claim 1.

22. The host cell as claimed in claim 20, is selected from CHO or BHK cell lines or their derivatives.

23. A process for production of proteins and peptides and variant thereof comprising:
a) constructing an expression vector as claimed in claim 1; and
b) transformation of said expression vector in suitable host cell which expresses the protein or peptide of interest.

24. A process for production of proteins and peptides and variants thereof comprising:
a) constructing an expression vector as claimed in claim 1;
b) transfection of said expression vector in a suitable host cell;
c) selecting suitable transfected host cell expressing a protein or peptide of interest;
d) suitable host cell selected in step (c) further retransfected with expression vector as claimed in claim 1; and
e) suitable retransfected host cell expressing a protein or peptide of interest.

25. The process as claimed in claim 23, wherein the expression vector has accession number MTCC 5655.

26. The process as claimed in claim 23, wherein the expression vector has accession number MTCC 5656.

27. The process as claimed in claim 23, wherein the expression vector has accession number MTCC 5657.

28. The process as claimed in claim 23, wherein the proteins and peptides are selected from tissue plasminogen activator, TNK-TPA, Darbepoietin, Erythropoietin, Insulin, GCSF, Interleukin, Tumor necrosis factor, Interferon, TNFR-IgGFc, Monoclonal antibodies such as rituximab, bevacizumab, adalimumab, trastuzumab and their fragments like Fc region, Fab, GLP-I, GLP-II, IGF-I, IGF-II, Platelet derived growth factor, FVII, FVIII, FIV and FXIII, exendin-3, exendin 4, hormones such as gonadotropic hormones selected from Follicle stimulating hormone, Human Chorionic Gonadotropin, and Human Luteinizing Hormone.


SEQUENCE LISTING (24 sequences)

SEQ ID NO: 1 - DNA, 324 bp, Artificial
  hybrid (Chimeric) INTRON
aattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagag

SEQ ID NO: 2 - DNA, 395 bp, Artificial
  Tripartite leader
gtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtacc

SEQ ID NO: 3 - DNA, 627 bp, Artificial
  Virus associated RNA genes I and II
ggtacctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccg

SEQ ID NO: 4 - DNA, 4584 bp, Artificial
  (Transcription assembly in clonig vector PMK) pZRC II
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggtacctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgaggaattccccggggatatcgtttaaacccacgatcgtggaagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 5 - DNA, 1668 bp, Artificial
  Chicken Lysozyme Matrix Attachment Region
gcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagctt

SEQ ID NO: 6 - DNA, 7932 bp, Artificial
  pZRC III plasmid pZRC II-1MAR(Sac) vector
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggtacctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgaggaattccccggggatatcgtttaaacccacgatcgtggaagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 7 - protein, 562 aa, Artificial
  Tenecteplase
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGNWSTAESGAECTNWQSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAAAAASPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 8 - DNA, 11138 bp, Artificial
  Plasmid having the Hygromycin transcription assembly (pZRC III- TNK- Hyg)
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttccggatctatccatcatggtggctttaccaacagtaccggaatgccaagctttttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagatcgcagatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgaggaattccaccatggacgccatgaagcggggactgtgctgtgtgctgctgctgtgtggcgccgtgttcgtgtcccctagccaggaaatccacgcccggttcagaaggggcgccaggtcctaccaagttatctgccgggacgaaaagacccagatgatctaccagcagcaccagtcctggctgcggcccgtgctgcggtccaaccgggtggagtactgctggtgcaactccggcagagcccagtgccactccgtgcctgtgaagtcctgctccgagcctagatgcttcaacggcggcacctgtcagcaggccctgtacttctccgacttcgtgtgccagtgccctgagggcttcgccggcaagtgctgcgagatcgacacccgggccacctgttacgaggaccagggcatctcctaccggggcaactggtctaccgccgagtctggcgccgagtgcaccaactggcagtcctccgccctggcccagaagccttactctggcagacggcctgacgccatcagactgggcctgggcaaccacaactactgccggaaccctgaccgggactccaagccttggtgctacgtgttcaaggccggcaagtactcctccgagttctgctccacccctgcctgctctgagggcaactccgactgctacttcggcaacggctccgcctacagaggcacccactccctgaccgagtccggcgcctcttgcctgccttggaactccatgatcctgatcggcaaggtgtacaccgcccagaacccttccgctcaggccctgggactcggaaagcacaattattgtcgcaatcccgacggcgacgccaaaccttggtgtcacgtgctgaagaaccggcggctgacatgggaatactgcgacgtgccttcctgctctacctgcggcctgcggcagtactcccagcctcagttccggatcaagggcggcctgttcgccgatatcgcctcccacccttggcaggccgccatcttcgccgctgctgccgcttctcctggcgagagattcctgtgcggcggcatcctgatctccagctgctggattctgtctgccgcccactgcttccaggaacggttccctcctcaccacctgaccgtgatcctgggccggacctacagagtcgtgcccggcgaggaagaacagaaattcgaggtggagaagtatatcgtgcacaaagagttcgacgacgacacctacgacaacgatatcgccctgctgcagctgaagtccgactcctccagatgcgcccaggaatcctccgtcgttcggaccgtgtgtctgccacctgccgacctgcagctgcctgactggaccgagtgcgagctgtccggctacggcaagcacgaggccctgtcccccttctactccgagcggctgaaagaagctcatgtacggctgtacccctctagccggtgcacctcccagcatctgctgaaccggaccgtgaccgacaacatgctgtgtgccggcgacaccagatctggcggccctcaggccaacctgcacgacgcctgccagggcgatagtggcggacctctcgtgtgcctcaacgacggcaggatgaccctcgtgggcatcatctcttggggcctgggctgtggccagaaagacgtgcctggcgtgtacaccaaagtgaccaactacctggactggatcagggacaacatgcggccttgatgagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 9 - protein, 193 aa, Artificial
  Darbepoetin fragment
MGVHECPAWLWLLLSLLSLPLGLPVLGAPPRLICDSRVLERYLLEAKEAENITTGCNETCSLNENITVPDTKVNFYAWKRMEVGQQAVEVWQGLALLSEAVLRGQALLVNSSQVNETLQLHVDKAVSGLRSLTTLLRALGAQKEAISPPDAASAAPLRTITADTFRKLFRVYSNFLRGKLKLYTGEACRTGDR

SEQ ID NO: 10 - DNA, 10023 bp, Artificial
  Plasmid having integrated Darbepoetin gene pZRC III- DARBE- Hyg
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttccggatctatccatcatggtggctttaccaacagtaccggaatgccaagctttttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagatcgcagatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgagccaccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtaatgaaacctgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccaggtgaacgagaccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 11 - protein, 489 aa, Artificial
  Etanercept
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 10911 bp, Artificial
  Plazmid pZRC III - Etanercept - Hyg
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttccggatctatccatcatggtggctttaccaacagtaccggaatgccaagctttttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagatcgcagatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgagccatggctccagtggctgtgtgggctgctctggctgtgggcctggaactgtgggccgctgctcacgctctgcctgctcaggtggccttcaccccttatgcccctgagcctggctccacctgcaggctgcgggagtactacgaccagaccgcccagatgtgctgctccaagtgctctcctggccagcacgccaaggtgttctgcaccaagacctccgacaccgtgtgcgactcttgcgaggactccacctacacccagctctggaactgggtgcccgagtgcctgtcctgcggctccagatgctcctccgaccaggtggagacacaggcctgtacacgggagcagaatcggatttgcacatgcaggcctggctggtactgcgccctgtccaagcaggaaggatgcaggctgtgcgcccctctgaggaagtgcagacctggcttcggcgtggctaggcctggcaccgagacatccgacgtcgtgtgcaagccttgtgcccctggcaccttctccaacaccacatcctccaccgacatctgccggcctcaccagatctgcaacgtggtggccatccctggcaacgccagcatggacgccgtgtgcacctccacctcccccaccagatctatggcccctggcgccgtgcatctgcctcagcctgtgtccacccggtcccagcatacccagcctacacctgagccctctaccgccccttctacctccttcctgctgcctatgggcccttctcctccagctgagggctctaccggcgacgagcctaagtcctgcgacaagacccacacctgtcccccctgccctgcccctgaactgctgggaggccccagcgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtgtccaacaaggctctgcctgcccccatcgaaaagaccatctccaaggccaagggccagccccgcgagcctcaggtgtacaccctgcctccttcccgggaggagatgaccaagaaccaggtgtccctgacctgtctggtcaagggcttctacccctccgatatcgccgtggaatgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagagcttatccctgtctcctggcaagtgataagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 13 - protein, 116 aa, Artificial
  FSH alpha subunit
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 14 - DNA, 599 bp, Artificial
  IRES gene fragment
gcggccgcgccgcactagaggaattccgcccctctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 15 - protein, 129 aa, Artificial
  FSH beta subunit
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 16 - DNA, 10801 bp, Artificial
  Plasmid comprising IRES- FSH beta subunits
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttccggatctatccatcatggtggctttaccaacagtaccggaatgccaagctttttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagatcgcagatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgagccaccatggactactaccggaagtacgccgccatcttcctggtgaccctgtccgtgttcctgcacgtgctgcactctgcccctgacgtgcaggactgccctgagtgcaccctgcaggaaaacccattcttcagccagcctggcgcccctatcctgcagtgcatgggctgctgcttctccagagcctaccctacccctctgcggtccaagaaaaccatgctggtgcagaaaaacgtgacctccgagtctacctgctgcgtggccaagtcctacaacagagtgaccgtgatgggcggcttcaaggtggagaaccacaccgcctgccactgctctacctgctactaccacaagtcctgatgagcggccgcgccgcactagaggaattccgcccctctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataacccgggccaccatgaagaccctgcagttcttcttcctgttctgctgctggaaggccatctgctgcaactcttgcgagctgaccaacatcacaatcgccatcgagaaagaggaatgccggttctgcatctccatcaacaccacttggtgcgccggctactgctacacccgcgacctggtgtacaaggaccctgcccggcctaagatccagaaaacctgcaccttcaaagaactggtgtacgagacagtgcgggtgccaggctgcgctcaccacgccgactccctgtacacctaccctgtggccacccagtgccactgcggcaagtgcgactccgactccaccgactgtaccgtgcggggcctgggcccttcttactgctccttcggcgagatgaaggaatgatgaccagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 17 - DNA, 10710 bp, Artificial
  Plasmid comprising Puromycin transcription assembly
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggtaccggatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccagatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgaggaattccaccatggacgccatgaagcggggactgtgctgtgtgctgctgctgtgtggcgccgtgttcgtgtcccctagccaggaaatccacgcccggttcagaaggggcgccaggtcctaccaagttatctgccgggacgaaaagacccagatgatctaccagcagcaccagtcctggctgcggcccgtgctgcggtccaaccgggtggagtactgctggtgcaactccggcagagcccagtgccactccgtgcctgtgaagtcctgctccgagcctagatgcttcaacggcggcacctgtcagcaggccctgtacttctccgacttcgtgtgccagtgccctgagggcttcgccggcaagtgctgcgagatcgacacccgggccacctgttacgaggaccagggcatctcctaccggggcaactggtctaccgccgagtctggcgccgagtgcaccaactggcagtcctccgccctggcccagaagccttactctggcagacggcctgacgccatcagactgggcctgggcaaccacaactactgccggaaccctgaccgggactccaagccttggtgctacgtgttcaaggccggcaagtactcctccgagttctgctccacccctgcctgctctgagggcaactccgactgctacttcggcaacggctccgcctacagaggcacccactccctgaccgagtccggcgcctcttgcctgccttggaactccatgatcctgatcggcaaggtgtacaccgcccagaacccttccgctcaggccctgggactcggaaagcacaattattgtcgcaatcccgacggcgacgccaaaccttggtgtcacgtgctgaagaaccggcggctgacatgggaatactgcgacgtgccttcctgctctacctgcggcctgcggcagtactcccagcctcagttccggatcaagggcggcctgttcgccgatatcgcctcccacccttggcaggccgccatcttcgccgctgctgccgcttctcctggcgagagattcctgtgcggcggcatcctgatctccagctgctggattctgtctgccgcccactgcttccaggaacggttccctcctcaccacctgaccgtgatcctgggccggacctacagagtcgtgcccggcgaggaagaacagaaattcgaggtggagaagtatatcgtgcacaaagagttcgacgacgacacctacgacaacgatatcgccctgctgcagctgaagtccgactcctccagatgcgcccaggaatcctccgtcgttcggaccgtgtgtctgccacctgccgacctgcagctgcctgactggaccgagtgcgagctgtccggctacggcaagcacgaggccctgtcccccttctactccgagcggctgaaagaagctcatgtacggctgtacccctctagccggtgcacctcccagcatctgctgaaccggaccgtgaccgacaacatgctgtgtgccggcgacaccagatctggcggccctcaggccaacctgcacgacgcctgccagggcgatagtggcggacctctcgtgtgcctcaacgacggcaggatgaccctcgtgggcatcatctcttggggcctgggctgtggccagaaagacgtgcctggcgtgtacaccaaagtgaccaactacctggactggatcagggacaacatgcggccttgatgagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 18 - DNA, 9595 bp, Artificial
  Plasmid comprising Darbepoetin gene fragment
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaagaggtaccggatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccagatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgagccaccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtaatgaaacctgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccaggtgaacgagaccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 19 - DNA, 10869 bp, Artificial
  Plasmid comprising Etanercept gene
gtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggatcctcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccagatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgacgcgtgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctcttccgcatcgctgtctgcgagggccagctgttggggtgagtactccctctcaaaagcgggcatgacttctgcgctaagaatgtcagtttccaaaaacgaggaggatttgatattcactggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgacgtaccaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcgcccgcactagaggctcgagccatggctccagtggctgtgtgggctgctctggctgtgggcctggaactgtgggccgctgctcacgctctgcctgctcaggtggccttcaccccttatgcccctgagcctggctccacctgcaggctgcgggagtactacgaccagaccgcccagatgtgctgctccaagtgctctcctggccagcacgccaaggtgttctgcaccaagacctccgacaccgtgtgcgactcttgcgaggactccacctacacccagctctggaactgggtgcccgagtgcctgtcctgcggctccagatgctcctccgaccaggtggagacacaggcctgtacacgggagcagaatcggatttgcacatgcaggcctggctggtactgcgccctgtccaagcaggaaggatgcaggctgtgcgcccctctgaggaagtgcagacctggcttcggcgtggctaggcctggcaccgagacatccgacgtcgtgtgcaagccttgtgcccctggcaccttctccaacaccacatcctccaccgacatctgccggcctcaccagatctgcaacgtggtggccatccctggcaacgccagcatggacgccgtgtgcacctccacctcccccaccagatctatggcccctggcgccgtgcatctgcctcagcctgtgtccacccggtcccagcatacccagcctacacctgagccctctaccgccccttctacctccttcctgctgcctatgggcccttctcctccagctgagggctctaccggcgacgagcctaagtcctgcgacaagacccacacctgtcccccctgccctgcccctgaactgctgggaggccccagcgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtgtccaacaaggctctgcctgcccccatcgaaaagaccatctccaaggccaagggccagccccgcgagcctcaggtgtacaccctgcctccttcccgggaggagatgaccaagaaccaggtgtccctgacctgtctggtcaagggcttctacccctccgatatcgccgtggaatgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagagcttatccctgtctcctggcaagtgataagcggccgcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacggagctcgcatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagcttgagctcatggcgcgcctaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 20 - DNA, 1710 bp, Artificial
  Tenecteplase (DNA sequence corresponding to Seq ID No. 7)
gaattccaccatggacgccatgaagcggggactgtgctgtgtgctgctgctgtgtggcgccgtgttcgtgtcccctagccaggaaatccacgcccggttcagaaggggcgccaggtcctaccaagttatctgccgggacgaaaagacccagatgatctaccagcagcaccagtcctggctgcggcccgtgctgcggtccaaccgggtggagtactgctggtgcaactccggcagagcccagtgccactccgtgcctgtgaagtcctgctccgagcctagatgcttcaacggcggcacctgtcagcaggccctgtacttctccgacttcgtgtgccagtgccctgagggcttcgccggcaagtgctgcgagatcgacacccgggccacctgttacgaggaccagggcatctcctaccggggcaactggtctaccgccgagtctggcgccgagtgcaccaactggcagtcctccgccctggcccagaagccttactctggcagacggcctgacgccatcagactgggcctgggcaaccacaactactgccggaaccctgaccgggactccaagccttggtgctacgtgttcaaggccggcaagtactcctccgagttctgctccacccctgcctgctctgagggcaactccgactgctacttcggcaacggctccgcctacagaggcacccactccctgaccgagtccggcgcctcttgcctgccttggaactccatgatcctgatcggcaaggtgtacaccgcccagaacccttccgctcaggccctgggactcggaaagcacaattattgtcgcaatcccgacggcgacgccaaaccttggtgtcacgtgctgaagaaccggcggctgacatgggaatactgcgacgtgccttcctgctctacctgcggcctgcggcagtactcccagcctcagttccggatcaagggcggcctgttcgccgatatcgcctcccacccttggcaggccgccatcttcgccgctgctgccgcttctcctggcgagagattcctgtgcggcggcatcctgatctccagctgctggattctgtctgccgcccactgcttccaggaacggttccctcctcaccacctgaccgtgatcctgggccggacctacagagtcgtgcccggcgaggaagaacagaaattcgaggtggagaagtatatcgtgcacaaagagttcgacgacgacacctacgacaacgatatcgccctgctgcagctgaagtccgactcctccagatgcgcccaggaatcctccgtcgttcggaccgtgtgtctgccacctgccgacctgcagctgcctgactggaccgagtgcgagctgtccggctacggcaagcacgaggccctgtcccccttctactccgagcggctgaaagaagctcatgtacggctgtacccctctagccggtgcacctcccagcatctgctgaaccggaccgtgaccgacaacatgctgtgtgccggcgacaccagatctggcggccctcaggccaacctgcacgacgcctgccagggcgatagtggcggacctctcgtgtgcctcaacgacggcaggatgaccctcgtgggcatcatctcttggggcctgggctgtggccagaaagacgtgcctggcgtgtacaccaaagtgaccaactacctggactggatcagggacaacatgcggccttgatgagcggccgc

SEQ ID NO: 21 - DNA, 601 bp, Artificial
  DNA sequence corresponding to Seq ID No. 9 Darbepoetin
ctcgagccaccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtaatgaaacctgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccaggtgaacgagaccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgagcggccgc

SEQ ID NO: 22 - DNA, 1489 bp, Artificial
  DNA sequence corresponding to Seq ID No. 11 Etanercept gene
ctcgagccatggctccagtggctgtgtgggctgctctggctgtgggcctggaactgtgggccgctgctcacgctctgcctgctcaggtggccttcaccccttatgcccctgagcctggctccacctgcaggctgcgggagtactacgaccagaccgcccagatgtgctgctccaagtgctctcctggccagcacgccaaggtgttctgcaccaagacctccgacaccgtgtgcgactcttgcgaggactccacctacacccagctctggaactgggtgcccgagtgcctgtcctgcggctccagatgctcctccgaccaggtggagacacaggcctgtacacgggagcagaatcggatttgcacatgcaggcctggctggtactgcgccctgtccaagcaggaaggatgcaggctgtgcgcccctctgaggaagtgcagacctggcttcggcgtggctaggcctggcaccgagacatccgacgtcgtgtgcaagccttgtgcccctggcaccttctccaacaccacatcctccaccgacatctgccggcctcaccagatctgcaacgtggtggccatccctggcaacgccagcatggacgccgtgtgcacctccacctcccccaccagatctatggcccctggcgccgtgcatctgcctcagcctgtgtccacccggtcccagcatacccagcctacacctgagccctctaccgccccttctacctccttcctgctgcctatgggcccttctcctccagctgagggctctaccggcgacgagcctaagtcctgcgacaagacccacacctgtcccccctgccctgcccctgaactgctgggaggccccagcgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtgtccaacaaggctctgcctgcccccatcgaaaagaccatctccaaggccaagggccagccccgcgagcctcaggtgtacaccctgcctccttcccgggaggagatgaccaagaaccaggtgtccctgacctgtctggtcaagggcttctacccctccgatatcgccgtggaatgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagagcttatccctgtctcctggcaagtgataagcggccgc

SEQ ID NO: 23 - DNA, 365 bp, Artificial
  DNA sequence corresponding to Seq ID No. 13 FSH alpha subunit
ctcgagccaccatggactactaccggaagtacgccgccatcttcctggtgaccctgtccgtgttcctgcacgtgctgcactctgcccctgacgtgcaggactgccctgagtgcaccctgcaggaaaacccattcttcagccagcctggcgcccctatcctgcagtgcatgggctgctgcttctccagagcctaccctacccctctgcggtccaagaaaaccatgctggtgcagaaaaacgtgacctccgagtctacctgctgcgtggccaagtcctacaacagagtgaccgtgatgggcggcttcaaggtggagaaccacaccgcctgccactgctctacctgctactaccacaagtcctgatga

SEQ ID NO: 24 - DNA, 415 bp, Artificial
  DNA sequence corresponding to Seq ID No. 15 FSH beta subunit
cccgggccaccatgaagaccctgcagttcttcttcctgttctgctgctggaaggccatctgctgcaactcttgcgagctgaccaacatcacaatcgccatcgagaaagaggaatgccggttctgcatctccatcaacaccacttggtgcgccggctactgctacacccgcgacctggtgtacaaggaccctgcccggcctaagatccagaaaacctgcaccttcaaagaactggtgtacgagacagtgcgggtgccaggctgcgctcaccacgccgactccctgtacacctaccctgtggccacccagtgccactgcggcaagtgcgactccgactccaccgactgtaccgtgcggggcctgggcccttcttactgctccttcggcgagatgaaggaatgatgaccagcggccgc